(19) United States
(12) Patent Application Publication
(10) Pub. No.: US 2023/0210724 A1
(43) Pub. Date: Jul. 6, 2023

(54) PORTABLE ELECTRONIC PILL DISPENSER WITH REPLACEABLE INSERT

(71) Applicant: Anthony J. Guarascio, Naples, FL (US)

(72) Inventor: Anthony J. Guarascio, Naples, FL (US)

(21) Appl. No.: 18/150,573

(22) Filed: Jan. 5, 2023

Related U.S. Application Data
(60) Provisional application No. 63/266,492, filed on Jan. 6, 2022.

(51) Int. Cl. A61J 7/00

(52) U.S. Cl. CPC A61J 7/0076; A61J 2205/60

(57) ABSTRACT

A pill dispenser includes a housing that surrounds a removable container for holding pills to be dispensed by the pill dispenser. The removable container includes a pill outlet that is accessible by a patient through the housing. In use, the medication only contacts the removable container and the pill outlet, so that when all of the pills within the removable container have been dispensed, the removable container may be removed from the container and safely disposed of before installing a new removable container. This allows the parts of the pill dispenser that have not contacted the medication to be reused.

### CROSS REFERENCE TO RELATED APPLICATIONS

This application claims the benefit of U.S. Provisional Application No. 63/266,492 filed Jan. 6, 2022 of which is hereby incorporated by reference.

### BACKGROUND

Controlled substances such as opioids and/or other narcotics may be prescribed to patients to help treat and manage pain. However, the use of these medicines is often heavily regulated to help prevent misuse of these substances. As an example, containers that have come into contact with a controlled substance may not be refilled or reused after the medicine within the container has been administered.

Pill dispensers may be used to assist a patient with the administration of medication. These pill dispensers may dispense the correct dose of medicine at the correct time to ensure that a patient is taking their prescription as intended. Some pill dispensers may include electronic components or other components that may be desired to be reused after dispensation of the medicine included in the pill dispenser. However, laws prohibiting reuse of containers that have come into contact with controlled substances may prevent these components from being reused. Therefore, improvement in this field is desired.

### SUMMARY

In a certain form, a pill dispenser includes a housing. A removable container including a container recess configured to hold pills to be dispensed by the pill dispenser is removably supported within the housing. A pill outlet is in communication with the container recess. The pill outlet includes a pill dispensing surface that is accessible from the exterior of the housing. Pills dispensed by the pill dispenser only contact the removable container and the pill outlet when the pill dispenser dispenses medication to a patient. The removable container and the pill outlet are configured to be removable from the housing to allow the removable container and the pill outlet to be disposed of after use and a new removable container filled with medication and pill outlet may be inserted into the housing.

### BRIEF DESCRIPTION OF THE DRAWINGS

FIG. 1 is a perspective view of a pill dispenser.

FIG. 2 is a left side view of the pill dispenser of FIG. 1.

FIG. 3 is a right side view of the pill dispenser of FIG. 1.

FIG. 4 is a perspective view of the pill dispenser of FIG. 1 with the housing and lid removed to show the interior of the pill dispenser.

FIG. 5 is a perspective view of a removable container of the pill dispenser of FIG. 1.

FIG. 6 is a top perspective view of the removable container of FIG. 5.

FIG. 7 is a bottom perspective view of the removable container of FIG. 5.

FIG. 8 is a front view of the pill dispenser of FIG. 1.

FIG. 9 is a rear view of the pill dispenser of FIG. 1.

FIG. 10 is a top view of the pill dispenser of FIG. 1.

FIG. 11 is a bottom view of the pill dispenser of FIG. 1.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

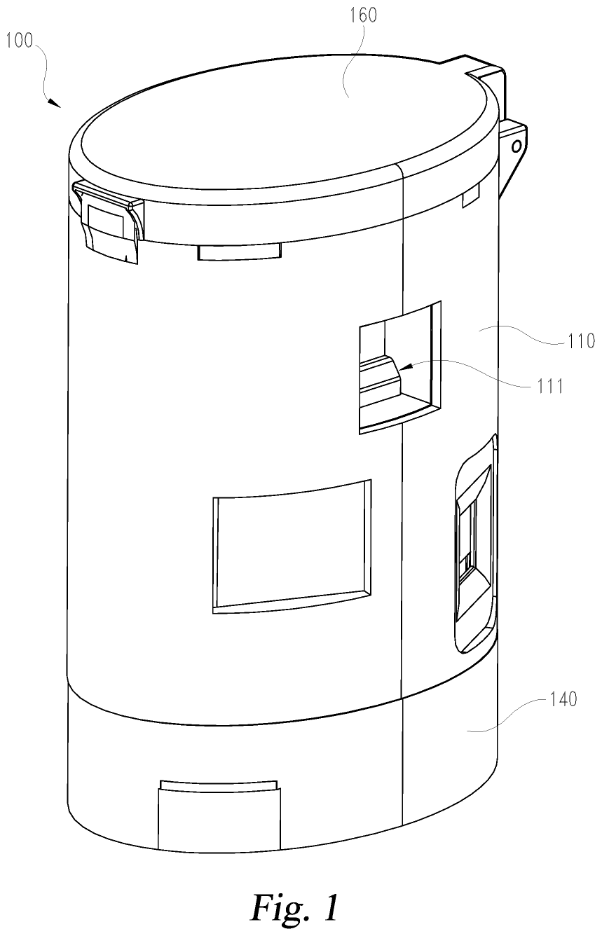

FIG. 1 illustrates a pill dispenser 100 that includes a housing 110. A bottom portion of the housing 110 is supported by a base 140. A lid 160 is connected to a top portion of the housing 110. In the embodiments shown, the lid is hinged to the housing 110, but in other examples, the lid 160 may be fit on the housing 110 by a friction fit or another suitable method. In some embodiments, the lid 160 is an optional feature and the housing 110 may have no lid 160.

In some embodiments, pill dispenser 100 is designed to be used with medications like narcotics that have strict rules regarding the reuse of devices that have come into contact with the medication. However, in other embodiments, any type of medication that may be dispensed from pill dispenser 100 may be used. The pill dispenser 100 is designed to include some portions that do not come into contact with the medication, and are therefore reusable, while other portions of the pill dispenser 100 are easily disposable.

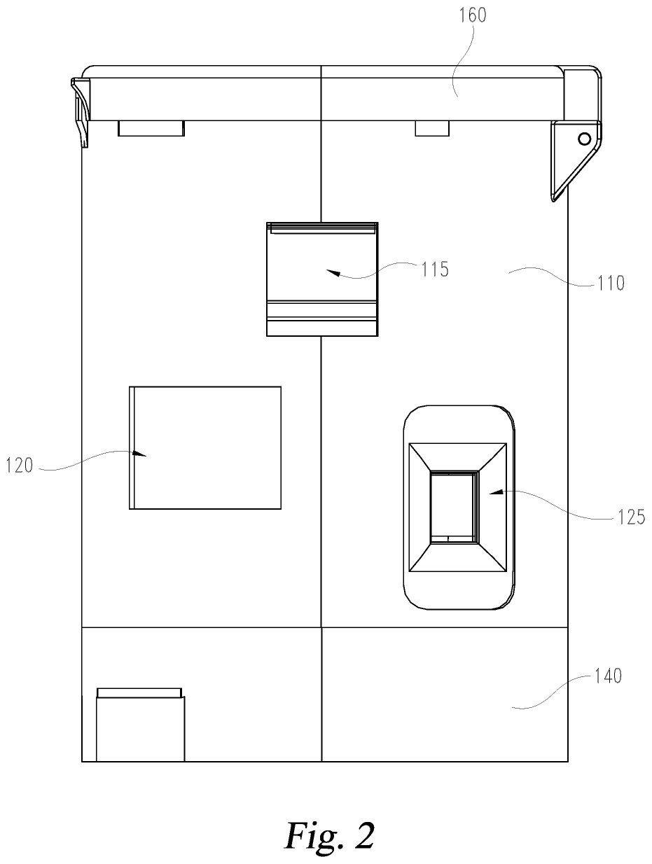

Various openings are defined through the housing 110, serving several different purposes. As shown in FIG. 2, an opening 111 for a pill outlet 115 is defined through a portion of the housing 110 on the front side of the pill dispenser 100. The pill outlet 115 provides a place for pills to be administered to a patient to be dispensed from the pill dispenser 100. The housing 110 may also include an LCD display 120. The LCD display 120 may be used to provide information regarding the pill dispenser 100 or may include patient data. For example, the LCD display 120 may show a user how many pills are left in the pill dispenser 100. In other embodiments, the LCD display 120 may show the last time a pill was dispensed or may include a clock or a timer that displays to a user the next time to take a pill. In still more embodiments, the LCD display 120 may also include the types of medication store in the pill dispenser 100 and/or information regarding the battery life left for the pill dispenser 100.

A biometric reader 125 is also positioned within housing 110. The biometric reader 125 may be used ensure that the correct patient is able to get pills from the pill dispenser 100. In some embodiments, the biometric reader 125 may be used to confirm identity. For example, the biometric reader 125 may be a finger print reader. In this example, a user must scan their finger print at the biometric reader 125 to have a pill or pills dispensed from the pill dispenser at the pill outlet 115.

In some embodiments, the biometric reader 125 may measure a biologic feature or a medical condition of the patient to ensure that the patient's vitals are in the correct range to be taking another dose of the medicine to be dispensed. As an example, the biometric reader 125 may measure blood oxygen levels, pulse, and/or perform a blood test to determine the medical condition of the patient. In other embodiments, other suitable medical conditions or vital signs may be monitored as desired to confirm that the patient should receive and take a dose of the medicine to be dispensed. In some embodiments, the pill dispenser 100 may be used as a combination of both identity confirmation and confirmation of medical suitability for receiving a dose of the medicine to be dispensed.

In some embodiments, the biometric reader 125 may not be integral with the pill dispenser 100. In these examples, a secondary device such as a ring, bracelet, watch, phone or other suitable monitoring device may be worn or carried by the patient, and information from the monitoring device may be delivered to the pill dispenser either wirelessly or by a wired connection.

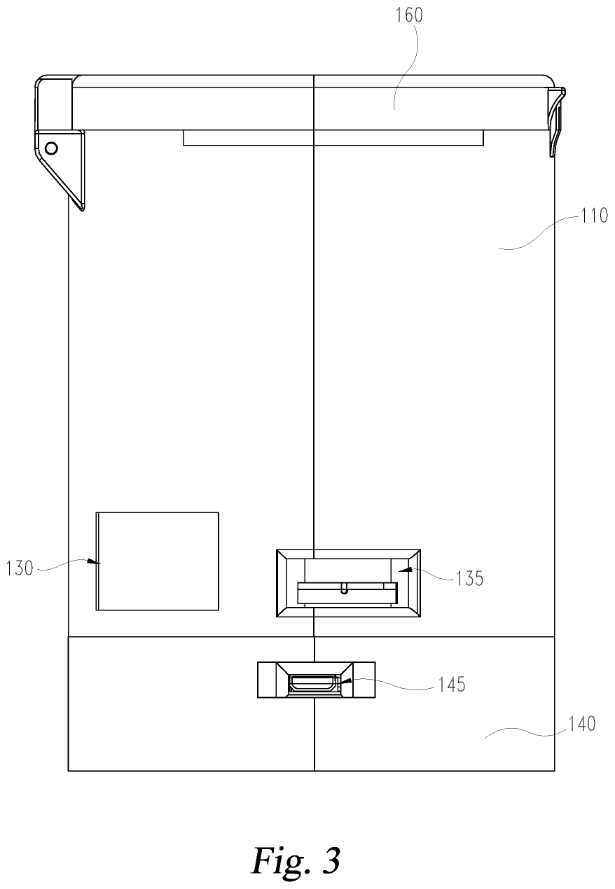

The back side of the pill dispenser 100 is illustrated in FIG. 3. An on/off switch 130 is included in the housing 110 on the back side of the pill dispenser 100. The on/off switch 130 may be used to turn off the pill dispenser 100 when the pill dispenser 100 is not in use to conserve energy. A sim port 135 is defined through the housing 110 and provides access to a controller positioned within the housing 110. The sim port 135 may receive a card or other suitable device that may provide instructions to the pill dispenser 100 regarding how to operate. Although in the embodiment shown, the sim port 135 is accessible externally from the housing 110, in other embodiments, the sim port 135 may be positioned within the housing 110. In these embodiments, the housing 110 must be removed to access the sim port 135.

A USB port 145 may be defined through the base 140 to allow connection to a USB cable that may be used to provide information to the pill dispenser 100 and/or may be used to provide power to the pill dispenser 100. Although the USB port 145 is shown through the base 140 of the pill dispenser 100 in FIG. 3, in other embodiments, the USB port 145 may extend through the housing 110 rather than the base 140.

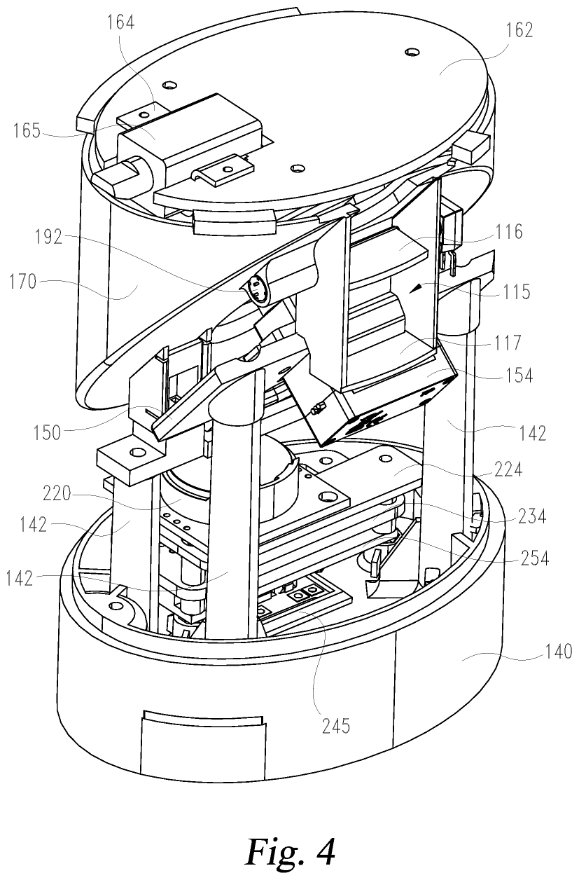

In FIG. 4, the pill dispenser 100 is shown without the housing 110 and the lid 160 to allow for a view of the inner workings of the pill dispenser 100. As shown, vertical supports 142 extend from the base 140. The vertical supports 142 support a servo holder 150 that includes a cutout that allows a servo 154 to extend through and be supported by the servo holder 150. The servo holder 150 also supports the pill outlet 115 which is connected to or integrally formed with a removable container 170 that is configured to hold pills to be dispensed by the pill dispenser 100. In some embodiments, the servo holder 150 may also provide support to the removable container 170.

Various electronic components used for operating the pill dispenser 100 and support surfaces for supporting these electronics are stored below the servo holder 150 and adjacent to the vertical supports 142. As an example, a battery 220 may be included on a battery support surface 224 for storing power to be used by the pill dispenser. In some embodiments, the battery 220 may provide power to a real time clock (RTC) device that can keep track of date and time regardless of whether the pill dispenser 100 is on or off or connected to a wireless network.

A sim holder may be positioned on a sim support surface 234 below the battery 220 and electronically connected to the sim port 135. The sim holder may receive a sim card to provide mobile network connectivity for the pill dispenser 100 to allow the pill dispenser 100 to communicate with remote devices and to provide information to third parties such as a doctor or a pharmacist who may monitor the dispensation of the pills in the pill dispenser 100.

Below the sim support surface 234 is a controller support surface 254 that provides support for a controller that is programmable to operate the pill dispenser 100. A power supply 245 that is in electronic communication with the USB port 145 may be positioned below the controller support surface 254. The power supply 245 may provide power to the other electronic components and power from the power supply may be stored in the battery 220.

A container cover 162 is positioned under the lid 160 and covers the removable container 170. A bracket 164 supports a lid lock 165 that is positioned within a recess defined through the container cover 162. The lid lock 165 may be used to lock the lid 160 in place with respect to the housing 110.

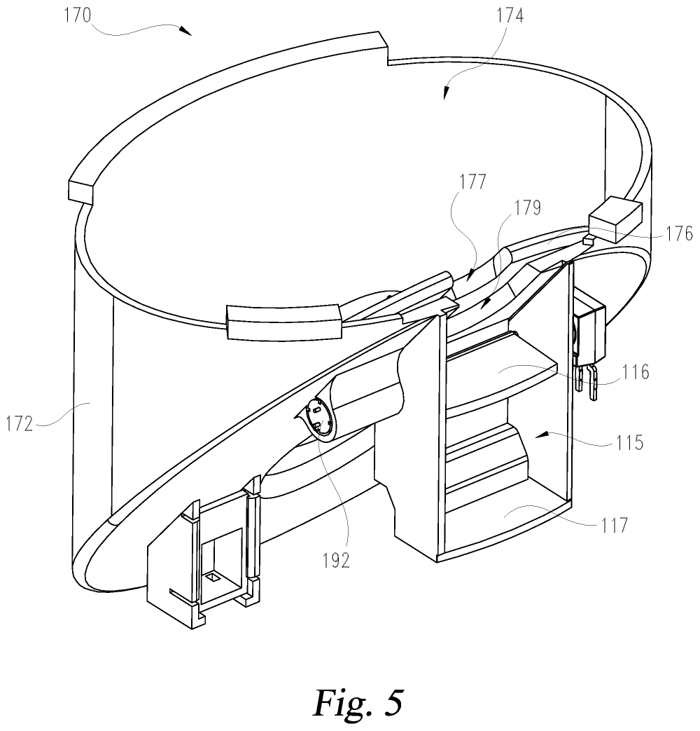

A perspective side view of the removable container 170 is shown in FIG. 5. The removable container 170 is shown to have a cylindrical shape in FIG. 5; however, in other embodiments, the removable container 170 may have any other suitable shape. As an example, the removable container may be rectangular or may have a square shape.

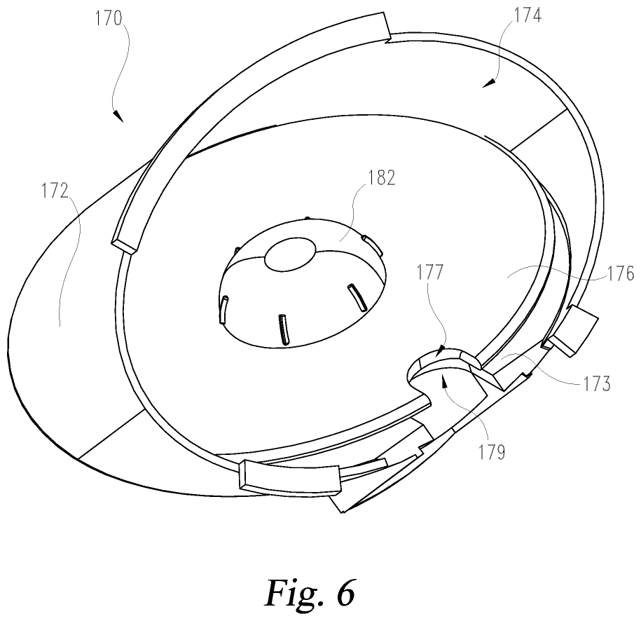

The removable container 170 includes a fixed plate 172 that forms the outer shell of the removable container 170. The fixed plate 172 may be connected to or integral with the structure of the pill outlet 115. A container recess 174 is defined through the fixed plate 172. The container recess 174 is configured to hold pills that are to be dispensed to the user of the pill dispenser 100. A movable plate 176 (see FIG. 6) is positioned within the container recess 174 and rests on an interior bottom surface 173 of the fixed plate 172. The movable plate 176 is mechanically coupled to the servo 154 so that operation of the servo 154 may cause the movable plate 176 to rotate upon the interior bottom surface 173 of the fixed plate 172.

A notch 177 is defined through a portion of the outer perimeter of the movable plate 176. When the notch 177 is aligned with the top of the pill outlet 115, one or more pills from within the container recess 174 may be delivered through the notch 177 into the pill outlet 115. In some embodiments, multiple notches 177 of different sizes may be defined through the movable plate 176 at different locations along the outer perimeter of the movable plate 176. The size of the notches 177 may correspond to different sized pills that are dispensed from the pill dispenser 100 to allow multiple pills to be stored within and dispensed from the pill dispenser 100.

A pill gate 116 is positioned within the pill outlet 115 and acts as a ledge that may hold one or more pills to be dispensed. As shown in FIG. 1, the pill gate 116 is positioned above the opening defined though the housing 110 by the pill outlet 115 so that a pill that is held on the pill gate 116 is not available to a user of the pill dispenser 100. A pill dispensing surface 117 is positioned below the pill holder and is configured to hold a pill that has been dispensed from the pill holder. As shown in FIG. 1, unlike the pill gate 116, the pill dispensing surface 117 is accessible to a user through the opening defined through the housing 110.

A fixed notch 179 is defined through the interior bottom surface 173 of the removable container 170. The fixed notch 179 is aligned with the position of the pill outlet 115 to allow access from the container recess 174 to the pill outlet 115 when the notch 177 is aligned with the fixed notch 179.

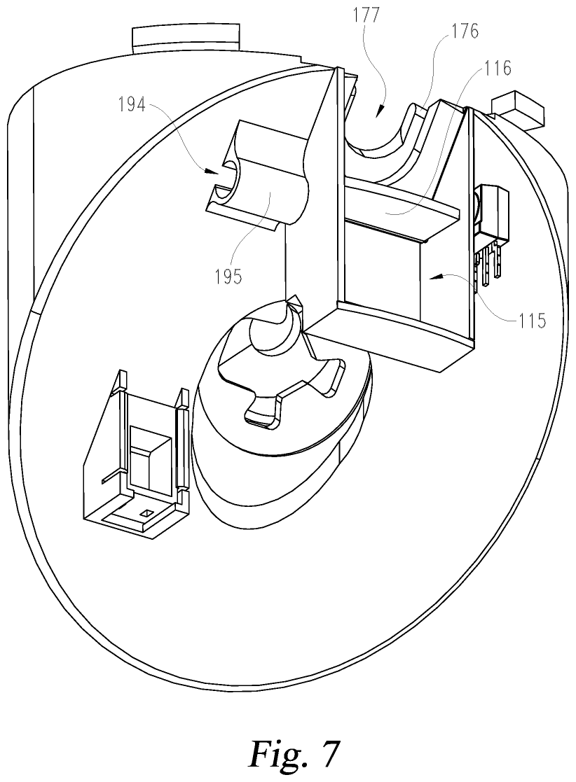

A motor 192 is inserted through a motor opening 194 defined through a motor housing 195 (see FIG. 7) that extends from the removable container 170. The motor 192 is coupled to the pill gate 116 so that actuation of the motor 192 causes the pill gate 116 to lower to allow a pill held on the pill gate 116 to fall to the pill dispensing surface 117 below. Further actuation of the motor 192 causes the pill gate 116 to raise so that the pill gate 116 is available to receive the next pill from the container recess 174.

A top perspective view of the removable container 170 is illustrated in FIG. 6. As shown, a screw cap 182 is positioned on the movable plate 176 and acts to secure the movable plate 176 to the interior bottom surface 173 of the removable container. To remove the movable plate 176 from the interior bottom surface 173, a user first unscrews and removes the screw cap 182 from the movable plate 176. The movable plate 176 may then be pulled upward, away from the interior bottom surface 173 and out of the container recess 174.

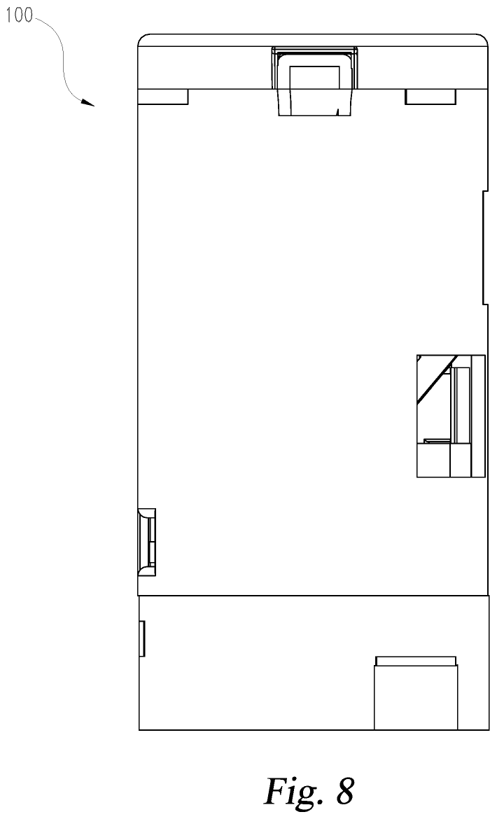

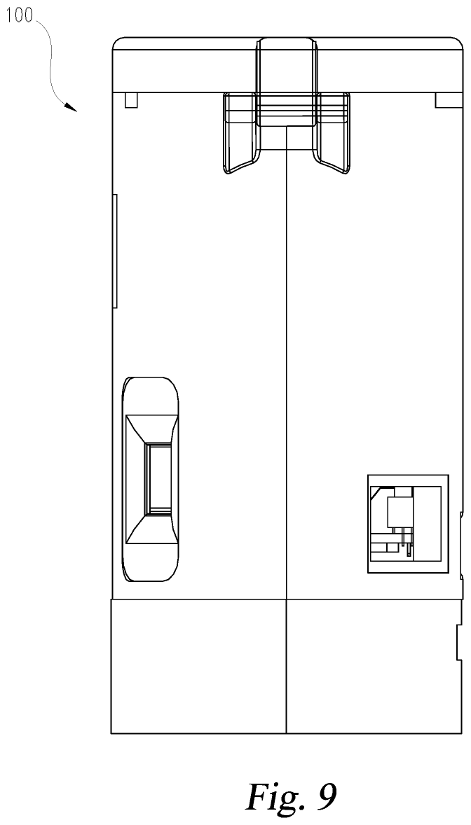

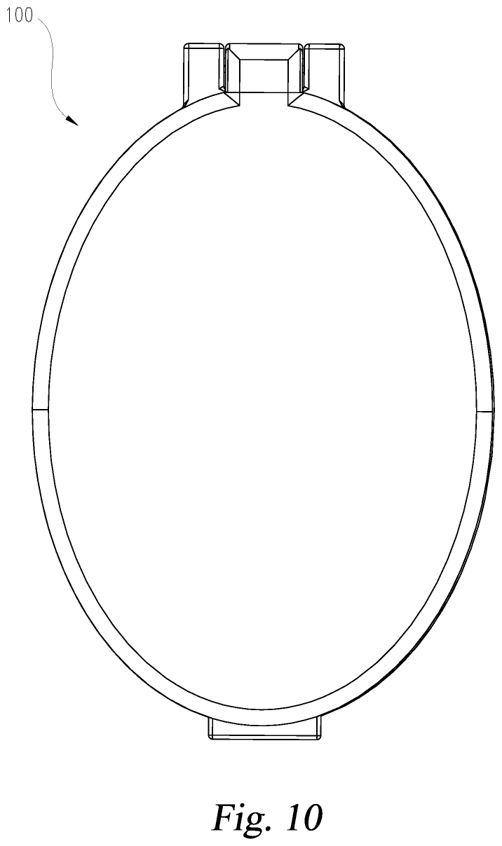

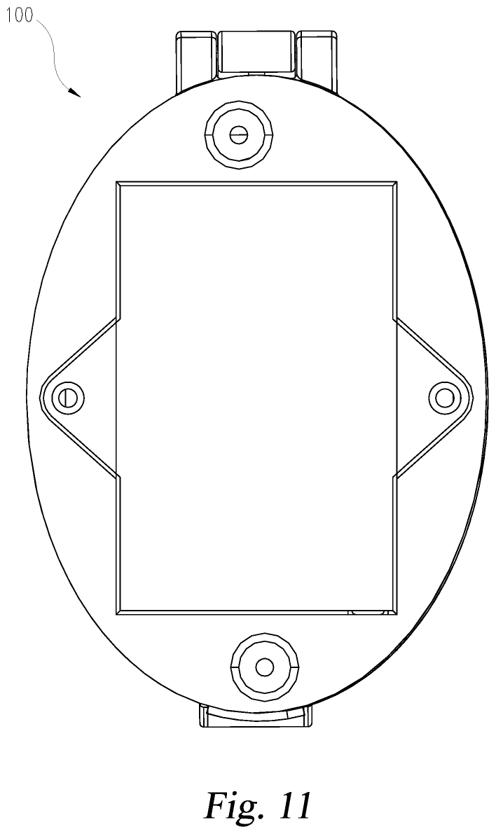

Additional views of the pill dispenser 100 are shown in FIGS. 8-11. For example, a front view of the pill dispenser is shown in FIG. 8. A rear view of the pill dispenser is illustrated in FIG. 9. A top view of the pill dispenser is illustrated in FIG. 10. A bottom view of the pill dispenser is illustrated in FIG. 11.

In some embodiments, a sensor or multiple sensors may be positioned within the removable container 170 and/or within the pill outlet 115 to monitor and confirm the number of pills that have been dispensed. If there were more or fewer pills dispensed than intended, the pill dispenser 100 may contact a health professional to alert the health professional of the discrepancy.

In operation, pills to be dispensed to a patient are inserted into the container recess 174 of the removable container 170, so that the pills rest on the movable plate 176. The servo 154 operates to rotate the movable plate 176 until the notch 177 is aligned with the fixed notch 179, so that a pill from within the container recess 174 may be dropped from the container recess 174 onto the pill gate 116. In some embodiments, the patient may then have to provide some kind of biometric signature, such as a fingerprint, at the biometric reader 125 to have the pill or pills dispensed onto the dispensing surface 117 of the pill outlet 115. Once the biometric signature has been authorized, the motor 192 is operated to cause the pill gate 116 to lower so that the pill or pills held on the pill gate 116 are dropped to the dispensing surface 117 below where the pills are accessible to the patient.

If multiple types of medication are held within the container recess 174, the movable plate 176 may have multiple notches 177. The sizes of the notches 177 may correspond to the sizes of the different pills that are held within the container recess 174. The servo 154 may be programmed to rotate the movable plate 176 to a desired position so that the correct notch 177 corresponding to the desired medication is aligned with the fixed notch 179 to dispense a specific type of pill at a certain time. In some instances, multiple pills may be held on pill gate 116 before being dispensed to the patient at the same time. In other instances, the different medications may be dispensed one at a time.

The removable container 170 is designed to be removed from the pill dispenser 100 when the removable container 170 runs out of pills to dispense to the patient. To remove the removable container 170, the lid 160 and the container cover 162 are removed from the housing 110 to allow access to the removable container 170. Then, the screw cap 182 is unscrewed from the movable plate 176. The movable plate 176 is then removed from the interior bottom surface 173 and pulled upward through the container recess 174. The removable container 170, including the pill outlet 115, may then be uncoupled from the servo holder 150 and separated from the pill dispenser 100. The motor 192 may be slid from the motor opening 194 of the motor housing 195 and saved to be reused with the next removable container 170.

The medication that is held within the removable container 170 is entirely contained within the removable container 170 throughout the entire process of loading the removable container 170 into the pill dispenser 100, operating the pill dispenser 100 to get the correct medication at the correct times of day, and dispensing the medication to the user. This allows the removable container 170 to be disposed of after all of the doses of medication within the removable container 170 have been provided to the patient. The other portions of the pill dispenser 100, such as the housing 110, the lid 160, the base 140, the servo 154 and servo holder 150, and the electronic control components may be reused because these portions of the pill dispenser 100 never come into contact with the medication throughout the dispensing process.

When a refill of the medication is desired, the empty removable container 170 is removed from the pill dispenser, and a new removable container 170 is inserted into the pill dispenser 100. The motor 192 from the empty removable container 170 may be reused by inserting the motor through the motor opening 194 in the motor housing 195 of the new removable container 170 and coupling the motor 192 to the new pill gate 116. In some embodiments, the new removable container 170 may be prefilled with the correct medication by a pharmacist or another medical professional or the removable container 170 may be prefilled at a distribution center. In other embodiments, the new removable container 170 may initially be empty and the patient may fill the removable container 170 with the necessary medication on their own.

In some embodiments, the pill dispenser 100 may have cellular network connectivity to allow the pill dispenser 100 to communicate with a database that includes information regarding the medication or medications being dispensed from the pill dispenser 100. In some embodiments, the pill dispenser 100 may communicate with a database that includes information regarding how patients use their medication to determine the pharmaceutical efficacy of the drugs dispensed from the pill dispenser 100.

In some embodiments, the pill dispenser 100 may monitor the amount of medication that is dispensed form the pill dispenser 100 over a certain amount of time. If the amount of medication dispensed exceed a preset limit, this may indicate a possible overdose, and the pill dispenser 100 may automatically contact emergency services to provide help for the patient.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

1. A pill dispenser comprising:
a housing;
a removable container including a container recess defined by a fixed plate, wherein said container recess is configured to hold pills to be dispensed by the pill dispenser;
a pill outlet extending from said fixed plate, wherein said pill outlet is in communication with the container recess, and wherein the pill outlet includes a pill dispensing surface that is accessible from the exterior of the housing;
wherein the removable container is removably supported within the housing;
wherein the pills dispensed by the pill dispenser only contact the removable container and the pill outlet when the pill dispenser dispenses medication to a patient; and,
wherein the removable container is configured to be removable from the housing to allow the removable container to be disposed of after use.

2. The pill dispenser of claim 1, further comprising:
a movable plate positioned within said container recess, wherein said movable plate is rotatable with respect to said fixed plate.

3. The pill dispenser of claim 2,
wherein said fixed plate includes a fixed notch and said movable plate includes a movable notch; and
wherein at least one pill held in said container recess is configured to be dispensed to said pill outlet when said movable notch is aligned with said fixed notch.

4. The pill dispenser of claim 2, further comprising:
a servo positioned within said housing; and
wherein said servo is operationally connected to said movable plate so that said servo is configured to rotate said movable plate with respect to said fixed plate.

5. The pill dispenser of claim 1,
wherein said pill outlet includes a pill gate positioned above said pill dispensing surface; and
wherein said pill gate is movable to allow a pill held on said pill gate to be dropped to said pill dispensing surface.

6. The pill dispenser of claim 1, further comprising:
a motor operated to move said pill gate between a raised position and a lower position.

7. The pill dispenser of claim 1, wherein said housing defines a pill outlet opening and
wherein said pill outlet is accessible through said pill outlet opening.

8. The pill dispenser of claim 1, further comprising:
a biometric reader positioned within said housing.

9. The pill dispenser of claim 1, further comprising:
a controller positioned within said housing.

10. A removable container for a pill dispenser comprising:
a fixed plate including an interior bottom surface, wherein said fixed plate forms an outer shell that defines a container recess;
a movable plate positioned within said container recess, wherein said movable plate is rotatable with respect to said interior bottom surface of said fixed plate;
a pill outlet attached to said fixed plate and in communication with the container recess, wherein the pill outlet includes a pill gate and a pill dispensing surface positioned below said pill gate;
wherein said pill outlet is in communication with said container recess through a movable plate notch defined through said movable plate.

11. The removable container of claim 10, wherein said movable plate is in contact with said interior bottom surface of said fixed plate.

12. The removable container of claim 10, wherein a fixed notch is defined through said interior bottom surface of said fixed plate, and wherein fixed notch is aligned with said pill outlet.

13. The removable container of claim 10, wherein said movable plate opening is a notch defined through defined through a portion of an outer perimeter of said movable plate.

14. The removable container of claim 10, wherein said pill outlet is integral to said fixed plate.

15. The removable container of claim 10, further comprising:
a screw cap positioned within said container recess; and
wherein said screw cap is configured to secure said movable plate to said fixed plate.

16. The removable container of claim 10, further comprising:
a motor housing positioned adjacent to said pill outlet, wherein said motor housing defines a motor opening;
a motor positioned within said motor opening, wherein said motor is configured to raise and lower said pill gate within said pill opening.

17. A method comprising:
inserting pills to be dispensed from a pill dispenser into a container recess defined in a removable container of said pill dispenser, wherein said removable container includes a pill outlet in communication with said container recess, a fixed plate including an interior bottom surface and a movable plate positioned within said container recess, and wherein said movable plate is rotatable with respect to said interior bottom surface of said fixed plate;
rotating said movable plate with respect to said fixed plate so that at least one of said pills in said container recess is dispensed to a pill gate of said pill outlet;
lowering said pill gate so that said at least one pill is dropped to a dispensing surface of said pill outlet;
removing said removable container from said pill dispenser.

18. The method of claim 17, further comprising:
providing a biometric signature to said pill dispenser prior to lowering said pill gate.

19. The method of claim 17, wherein a motor is operated to move said pill gate of said pill outlet between a raised position and a lowered position.

20. The method of claim 17,
wherein said fixed plate includes a fixed notch and said movable plate includes a movable notch; and
wherein said at least one of said pills in said container recess is dispensed to said pill gate when said movable notch is aligned with said fixed notch.
